(19) United States
(12) Patent Application Publication
(10) Pub. No.: US 2012/0283502 A1
(43) Pub. Date: Nov. 8, 2012

(54) ULTRASOUND NEUROMODULATION TREATMENT OF DEPRESSION AND BIPOLAR DISORDER

(76) Inventors: David J. MISHELEVICH, Playa del Rey, CA (US); Tomo SATO, Roanoke, VA (US); William J. TYLER, Roanoke, VA (US); Daniel Z. WETMORE, San Francisco, CA (US)

(21) Appl. No.: 13/426,424

(22) Filed: Mar. 21, 2012

Related U.S. Application Data
(60) Provisional application No. 61/454,738, filed on Mar. 21, 2011.

(51) Int. Cl. A61N 7/00; A61N 5/00

(52) U.S. Cl. 600/2; 601/2; 600/1

(57) ABSTRACT

Disclosed are methods and systems and methods for non-invasive neuromodulation using ultrasound to treat depression, bipolar disorder, and other mood disorders. Also disclosed are methods and systems and methods for non-invasive neuromodulation using ultrasound to affect the mood or emotional state of a subject or user. The neuromodulation can produce acute or long-term effects. The latter occur through Long-Term Potentiation (LTP) or Long-Term Depression (LTD) via training. Included is control of direction of the energy emission, intensity, frequency, pulse duration, and phase/intensity relationships to targeting and accomplishing up regulation and/or down regulation.

### CROSS REFERENCE TO RELATED APPLICATIONS

This provisional patent application claims priority to provisional patent application No. 61/454,738, filed Mar. 21, 2011, titled “ULTRASOUND NEUROMODULATION TREATMENT OF DEPRESSION AND BIPOLAR DISORDER”. The disclosures of this patent application are herein incorporated by reference in their entirety.

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Ultrasound Neuromodulation including one or more ultrasound sources for neuromodulation of target deep brain regions to up-regulate or down-regulate neural activity. The present invention relates to methods and systems for achieving effective neuromodulation by transcranial ultrasound (bioTU) for the treatment of depression, bipolar disorder, and other mood disorders. The present invention also relates to methods and systems for effective neuromodulation by bioTU to affect mood or emotional state in a subject.

### BACKGROUND OF THE INVENTION

It has been demonstrated that focused ultrasound directed at neural structures can stimulate those structures. If neural activity is increased or excited, the neural structure is said to be up regulated; if neural activated is decreased or inhibited, the neural structure is said to be down regulated. Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

Ultrasound (US) has been used for many medical applications, and is generally known as cyclic sound pressure with a frequency greater than the upper limit of human hearing. The production of ultrasound is used in many different fields, typically to penetrate a medium and measure the reflection signature or to supply focused energy. For example, the reflection signature can reveal details about the inner structure of the medium. A well-known application of this technique is its use in sonography to produce a picture of a fetus in a womb. There are other applications which may provide therapeutic effects, such as lithotripsy for ablation of kidney stones or high-intensity focused ultrasound for thermal ablation of brain tumors. An important benefit of ultrasound therapy is its non-invasive nature. US waveforms can be defined by their acoustic frequency, intensity, waveform duration, and other parameters that vary the timecourse of acoustic waves in a target tissue. US waveforms based on repeated pulses less than about 1 second are generally referred to as pulsed ultrasound and are repeated at a rate equivalent to the pulse repetition frequency. Tone bursts that extend for about 1 second or longer—though, strictly speaking, also pulses—are often referred to as continuous wave (CW).

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested.

Alternative mechanisms for the effects of ultrasound may be discovered as well. In fact, multiple mechanisms may come into play, but, in any case, this would not effect this invention.

Neurons are mechanically sensitive and can act as a piezoelectric material by converting a mechanical displacement into electrical currents or membrane polarization. Several potential mechanisms for the conversion of mechanical energy into neuronal activity have been proposed. Stretch-induced activation or inactivation of ion channels is one mechanism for converting mechanical force into currents that modulate neuronal activity. Mechanosensitive ion channels convert mechanical force into an electrical signal and contribute to transduction of hearing and touch (Sukharev and Corey, 2004). Ion channels and receptors that mediate their primary physiological effect through non-mechanical means are also sensitive to mechanical forces. Reversible activation and inactivation responses to stretch have been observed in recombinant systems for voltage-gated Na+, Ca2+ (L-type and N-type), and K+ ion channels, as well as for the hyperpolarization-activated channel, HCN (Morris and Juranka, 2007a; Morris and Juranka, 2007b). One mechanism of stretch-induced effects in ion channels is thought to be caused by linear spring properties endowed by their structure. An additional or alternative mechanism of stretch-induced effects in ion channels may relate to mechanical effects on cytoskeletal proteins such as actin or tubulin that could then be transduced to membrane-bound ion channels through the cytoskeletal structure.

Flexoelectric effects are a second mechanism for converting mechanical energy into changes in neuronal activity. Flexoelectricity was first discovered in the context of liquid crystals. Petrov described flexoelectricity in the context of biological membranes as “a phenomenon of curvature-induced electric polarization of a liquid crystal membrane, in which the molecules of the membrane are uniaxially orientated. Curvature of a membrane bilayer splays the uniaxial orientation of the molecules (lipids, proteins) that it contains and imposes a polar symmetry, such that on one side of the membrane the molecules are moved apart whereas on the other side they are moved closer together. Flexoelectricity results from the resultant electrical polarization of the membrane” (Petrov et al., 1993). Flexoelectric effects in hair cell stereocilia in the inner ear are thought to play a role in hearing by converting membrane depolarization into changes in the mechanical properties of stereocilia (Brenenman and Rabbitt, 2009). Alternatively, flexoelectric effects can operate in the reverse direction in which mechanical energy is converted into membrane polarization. Thermodynamic investigations of lipid-phase transitions have shown that mechanical waves can be adiabatically propagated through lipid monolayers and bilayers, as well as neuronal membranes to influence fluidity and excitability (Griesbauer et al., 2009; Heimburg, 2010). Notably, such sound wave propagation in pure lipid membranes has been estimated to produce depolarizing potentials ranging from 1 to 50 mV with negligible heat generation (˜0.01 K) (Griesbauer et al., 2009), potentially via a flexoelectric effect. In this manner, mechanical energy delivered by an acoustic wave can cause membrane polarization and affect voltage-gated channels and thus neuronal activity.

Another potential mechanism for neuromodulation by ultrasound is by causing changes in blood flow through mechanical and/or thermal effects.

Neuromodulation of the brain by ultrasound has been shown in animals using transcranial ultrasound for neuromodulation (bioTU). Other transcranial ultrasound based techniques use a combination of parameters, including high intensities (greater than about 1 W/cm2) and/or high acoustic frequencies (greater than about 1 MHz) and/or pulsing and waveform parameters, that disrupt or otherwise affect neuronal cell populations so that they do not function properly and/or heat tissue (bone tissue or soft tissue) so as to damage or ablate tissue. bioTU employs a combination of parameters that transmits mechanical energy through the skull to its target in the brain without causing significant thermal or mechanical damage and induces neuromodulation primarily through mechanical means.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to affect a multi-beam output. These transducers are coordinated by a computer and used in conjunction with an imaging system, preferable an fMRI (functional Magnetic Resonance Imaging), but possibly a PET (Positron Emission Tomography) or, V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. According to the Bystritsky patent '861, differences in FUP phase, frequency, and amplitude produce different neural effects. Low frequencies (defined as below 300 Hz) are inhibitory. According to the Bystritsky patent '861, high frequencies (defined as being in the range of 500 Hz to 5 MHz are excitatory and activate neural circuits. This works whether the target is gray or white matter. Repeated sessions result in long-term effects. The cap and transducers to be employed are preferably made of non-ferrous material to reduce image distortion in fMRI imaging. The Bystritsky patent '861, noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

Deisseroth and Schneider (U.S. patent application Ser. No. 12/263,026 published as US 2009/0112133 A1, Apr. 30, 2009) describe an alternative approach in which modifications of neural transmission patterns between neural structures and/or regions are described using ultrasound (including use of a curved transducer and a lens) or RF. The impact of Long-Term Potentiation (LTP) and Long-Term Depression (LTD) for durable effects is emphasized. It is noted that ultrasound produces stimulation by both thermal and mechanical impacts. The use of ionizing radiation also appears in the claims.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February; 24(2):275-83 and Clement G T, Hynynen K (2002) A non-invasive method for focusing ultrasound through the human skull. Phys Med Biol 47: 1219-1236). Ultrasound can be focused to 0.5 to 2 mm as TMS to 1 cm at best.

Recent research and disclosures have described the use of bioTU to activate, inhibit, or modulate neuronal activity (Bystritsky et al., 2011; Tufail et al., 2010; Tufail et al., 2011; Tyler et al., 2008; Yang et al., 2011; Yoo et al., 2011; Zaghi et al., 2010), the full disclosures of which are incorporated herein by reference. Also see U.S. Pat. No. 7,283,861 and US patent applications 20070299370, 20110092800 titled “Methods for modifying currents in neuronal circuits” by inventor Alexander Bystritsky; patent applications by one or more of the named inventors of this submission: patent application Ser. Nos. 13/003,853 (Publication number: US 2011/0178441 A1) titled “Methods and devices for modulating cellular activity using ultrasound” and PCT/US2010/055527 (Publication number: WO/2011/057028) titled “Devices and methods for modulating brain activity”, and patent application [Is there a number assigned yet?] titled “Improvement of Direct Communication”; and US patent applications by inventor David J. Mishelevich: Ser. No. 12/917,236 (Publication number: US 2011/0082326 A1) titled “TREATMENT OF CLINICAL APPLICATIONS WITH NEUROMODULATION”; Ser. No. 12/940,052 (Publication number: US 2011/0112394 A1) titled “NEUROMODULATION OF DEEP-BRAIN TARGETS USING FOCUSED ULTRASOUND”; Ser. No. 12/958,411 (Publication number: US 2011/0130615 A1) titled “MULTI-MODALITY NEUROMODULATION OF BRAIN TARGETS”; Ser. No. 13/007,626 (Publication number: US 2011/0178442 A1) titled “PATIENT FEEDBACK FOR CONTROL OF ULTRASOUND DEEP-BRAIN NEUROMODULATION”; Ser. No. 13/020,016 (Publication number: US 2011/0190668 A1) titled “ULTRASOUND NEUROMODULATION OF THE SPHENOPALATINE GANGLION”; Ser. No. 13/021,785 (Publication number: US 2011/0196267 A1) titled “ULTRASOUND NEUROMODULATION OF THE OCCIPUT”; Ser. No. 13/031,192 (Publication number: US 2011/0208094 A1) titled “ULTRASOUND NEUROMODULATION OF THE RETICULAR ACTIVATING SYSTEM”; Ser. No. 13/035,962 (Publication number: US 2011/0213200 A1) titled “ORGASMATRON VIA DEEP-BRAIN NEUROMODULATION”; and Ser. No. 13/098,473 (Publication number: US 2011/0270138) titled “Ultrasound Macro Pulse And Micro Pulse Shapes For Neuromodulation”, the full disclosures of which are incorporated herein by reference). The actual mechanisms underlying bioTU have not been fully elucidated. However, one confirmed mechanism for bioTU stimulation of electrical activity in neurons is by activating voltage-gated sodium channels and voltage-gated calcium channels (Tyler et al., 2008). bioTU can induce SNARE-mediated vesicle release and synaptic transmission (Tyler et al., 2008). In contrast to US waves with higher intensities, bioTU does not lead to significant tissue heating in the targeted brain region (Tufail et al., 2010). bioTU activates c-fos and does not disrupt the blood brain barrier (Tufail et al., 2010).

An appropriate ultrasound stimulation protocol must be delivered in order to induce changes in the brain via bioTU. The temporal pattern of ultrasound vibration delivered to the brain affects the induced neuromodulation. The temporal pattern of ultrasound waveforms may also affect the nature of the induced neuromodulatory effect such as neuromodulation (which may be mediated by a change in the excitability of neuronal circuits), stimulation of neuronal activity, or inhibition of neuronal activity. Effective and ineffective parameters for ultrasound neuromodulation have been described previously. Tyler et al. used the genetically encoded pH-sensitive indicator synaptopHluorin to monitor synaptic vesicle release in CA1 pyramidal neurons in acute hippocampal slices while varying parameters of pulsed ultrasound; also see patent application Ser. Nos. 13/003,853 (Publication number: US 2011/0178441 A1) titled “Methods and devices for modulating cellular activity using ultrasound” and PCT/US2010/055527 (Publication number: WO/2011/057028) titled “Devices and methods for modulating brain activity” by inventor Tyler).

Methods and systems for generating ultrasound waveforms for ultrasound neuromodulation have been described. Patent application Ser. No. 13/098,473 (Publication number: US 2011/0270138) by inventor Mishelevich titled “Ultrasound Macro Pulse And Micro Pulse Shapes For Neuromodulation” teaches superimposing pulse trains on the base ultrasound carrier and heterogeneous patterns of pulse shaping with sine waves, square waves, triangular waves, or arbitrarily shaped waves. Patent application Ser. No. 13/003,853 (Publication number: US 2011/0178441 A1) by inventor Tyler titled “Methods and devices for modulating cellular activity using ultrasound” teaches ultrasound waveform repetition, varying the length and frequency of ultrasound pulses; varying the one or more dominant acoustic frequencies of ultrasound; shaping ultrasound pulses by a sine wave, square wave, saw-tooth pattern, arbitrary waveform; and combinations of one or more waveform. Patent application PCT/US2010/055527 (Publication number: WO/2011/057028 [What is the US #?]) by inventor Tyler titled “Devices and methods for modulating brain activity” teaches ultrasound waveforms shaped as sine waves having a single ultrasound frequency and other oscillating shapes such as square waves, sawtooth waves, triangle waves, or spikes, or ramps, or a pulse that includes multiple ultrasound frequencies composed of beat frequencies, harmonics, or a combination of frequencies generated by constructive or deconstructive interference techniques, or some or all of the aforementioned. Patent application [NT3; (recent filing, communications patent) number] by inventors Tyler et al. titled “Improvement of direct communication” teaches bioTU ultrasound waveforms of any type known in the art including but not limited to amplitude modulated waveforms, tone-bursts, pulsed waveforms, and continuous waveforms. The “Improvement of direct communication” patent application also teaches bioTU repetition frequency that may be fixed or variable. Variable bioTU repetition frequency values taught may be random, pseudo-random, ramped, or otherwise modulated.

Because of the utility of ultrasound in the neuromodulation of deep-brain structures, it would be both logical and desirable to apply it to the treatment of depression and bipolar disorder.

### SUMMARY OF THE INVENTION

It is the purpose of this invention to provide methods and systems for non-invasive neuromodulation using transcranial ultrasound to treat depression (including Major Depressive Disorder (MDD)), bipolar disorder, and other mood disorders. It is also the purpose of this invention to provide methods and systems for non-invasive neuromodulation using transcranial ultrasound to affect mood or emotional state in a subject. Such neuromodulation can produce acute effects or long-lasting effects that may be due to Long-Term Potentiation (LTP) and/or Long-Term Depression (LTD) in neuronal circuits. Included is control of direction of the energy emission, intensity, frequency, pulse duration, and phase/intensity relationships to target appropriate one or more brain regions and achieve appropriate neuromodulation to induce the intended effect on a mood disorder or on the emotional state of a subject. The effect may be accomplished via up-regulation and/or down-regulation in the brain. Use of ancillary monitoring or imaging to provide feedback is optional. In embodiments where concurrent imaging is performed, the device of the invention is constructed of non-ferrous material.

Multiple targets can be neuromodulated singly or in groups to treat depression, bipolar depression, or other mood disorder. Multiple targets can be neuromodulated singly or in groups to affect the mood or emotional state of a subject. To accomplish the treatment or modulation of mood or emotional state, in some cases the neural targets will be up regulated and in some cases down regulated, depending on the given neural target and intended effect. Targets have been identified by such methods as PET imaging, fMRI imaging, and clinical response to Transcranial Magnetic Stimulation (TMS). In various embodiments of the invention the targeted brain region and form of neuromodulation include one or more chosen from the following list: the Left Prefrontal Cortex would be up regulated (George, M. S., Wassermann, E. M., Williams, W. A., Callahan A., Ketter, T. A., Basser, P., Hallett, M., and R. M. Post, “Daily repetitive transcranial magnetic stimulation (rTMS) improves mood in depression,” Neuroreport 1995; 6:1853-1856), the Right Prefrontal Cortex down regulated (Menkes, D. L., Bodnar, P., Ballesteros, R. A., and M. R. Swenson, “Right frontal lobe slow frequency repetitive transcranial magnetic stimulation (SF r-TMS) is an effective treatment for depression: a case-control pilot study of safety and efficacy,”0.1 Neurol Neurosurg Psychiatry 1999; 67:113-115), Orbito-Frontal Cortex (OFC) (Lee, Seong, et al., 2007 (Lee, B. T., Seong, Whi Cho, Hyung, Soo Khang, Lee. B. C., Choi I. G., Lyoo, I. K., and B. J. Ham, “The neural substrates of affective processing toward positive and negative affective pictures in patients with major depressive disorder,” Prog Neuropsychopharmacol Biol Psychiatry. 2007 Oct. 1; 31(7):1487-92. Epub 2007 Jul. 5)) would be up regulated, the Anterior Cingulate Cortex (ACC) would be up regulated (Lee, Seong, et al., 2007), the Subgenu Cingulate (Johansen-Berg, H., Gutman, D. A., Behrens, T. E., Matthews, P. M., Rushworth, M. F., Katz, I E., Lozano, A. M., and H. S. Mayberg, “Anatomical connectivity of the subgenual cingulate region targeted with deep brain stimulation for treatment-resistant depression,” Cereb Cortex. 2008 June; 18(6):1374-83. Epub 2007 Oct. 10) down regulated, the Right Insula (Lee, Seong, et al., 2007) up regulated, the left Insula (Lee, Seong, et al., 2007) down regulated, the Nucleus Accumbens (Hauptman, J. S., DeSalles, A. A., Espinoza, R., Sedrak, M., and W. Ishida, “Potential surgical targets for deep brain stimulation in treatment-resistant depression.,” Neurosurg Focus. 2008; 25(1):E3) up regulated, the Caudate Nucleus (Lee, Seok et al, 2008 (Lee, B. T., Seok, J. H., Lee, B. C., Cho, S. W., Yoon, B. J., Lee, K. U., Chae, J. H., Choi, L. G., and B. J. Ham, “Neural correlates of affective processing in response to sad and angry facial stimuli in patients with major depressive disorder, “Prog Neuropsychopharmacol Biol Psychiatry. 2008 Apr. 1; 32(3):778-85. Epub 2007 Dec. 23)) up regulated, the Amygdala (Lee, Seong, et al., 2007) down regulated, and the Hippocampus (Lee, Seok et al, 2008) up regulated. The specific targets and/or whether the given target is up regulated or down regulated, can depend on the individual patient or subject and relationships of up regulation and down regulation among targets, and the patterns of stimulation applied to the targets. In some cases neuromodulation will be bilateral and in others unilateral.

When considering other treatment modalities, if targets care appropriately modulated by Transcranial Magnetic Stimulation, the same targets would be used for ultrasound neuromodulation. The targeting can be done with one or more of known external landmarks, an atlas-based approach or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

While ultrasound can be focused down to a diameter on the order of one to a few millimeters (depending on the frequency), whether such a tight focus is required depends on the conformation of the neural target.
Bachtold, M. R., Rinaldi, P. C., Jones, J. P., Reines, F., and Price, L. R. (1998). Focused ultrasound modifications of neural circuit activity in a mammalian brain. Ultrasound in medicine &amp; biology 24, 557-565,
Breneman, K. D., and Rabbitt, R. D. (2009). Piezo- and Flexoelectric Membrane Materials Underlie Fast Biological Motors in the Ear. Materials Research Society symposia proceedings Materials Research Society 1186E.
Bystritsky, A., Korb, A. S., Douglas, P. K., Cohen, M. S., Melega, W. P., Mulgaonkar, A. P., DeSalles, A., Min, B.-K., and Yoo, S.-S. (2011). A review of low-intensity ifocused ultrasound pulsation. Brain stimulation 4, 125-136.
Dalecki, D. (2004). Mechanical bioeffects of ultrasound. Annual review of biornedical engineering 6, 229-248.
Gavrilov, L. R., Gersuni, G. V., Ilyinsky, O. B., Sirotyuk, M. G., Tsirulnikov, E. M., and Shchekanov, E. E. (1976). The effect of focused ultrasound on the skin and deep nerve structures of man and animal. Progress in brain research 43, 279-292.
Griesbauer, J., Wixfbrth, A., and Schneider, M. F. (2009). Wave Propagation in Lipid Monolayers. Biophysical Journal 97, 2710-2716.
Heimburg, T. (2010). Lipid ion channels. Biophysical chemistry 150, 2-22.
Hynynen, K., and Clement, G. (2007). Clinical applications of focused ultrasound—the brain. International journal of hyperthermia: the official journal of European Society for Hyperthermic Oncology, North American Hyperthermia Group 23, 193-202.
Hynynen, K., Clement, G. T., McDannold, N., Vykhodtseva, N., King, R, White, P. J., Vitek, S., and Jolesz, F. A. (2004). 500-element ultrasound phased array system for noninvasive focal surgery of the brain: a preliminary rabbit study with ex vivo human skulls. Magnetic resonance in medicine: official journal of the Society of Magnetic Resonance in Medicine/Society of Magnetic Resonance in Medicine 52, 100-107.
Mihran, R. T., Barnes, F. S., and Wachtel, H. (1990). Temporally-specific modification of nmyelinated axon excitability in vitro following a single ultrasound pulse. Ultrasound in medicine &amp; biology 16, 297-309.
Morris, C., and Juranka, P. (2007a). Current Topics in Membranes. 59, 297-338.
Morris, C. E., and Juranka., P. F. (2007b). Nay channel mechanosensitivity: activation and inactivation accelerate reversibly with stretch. Biophysical Journal 93, 822-833.
O'Brien, W. D. (2007). Ultrasound-biophysics mechanisms. Progress in biophysics and molecular biology 93, 212-255.
Petrov, A. G., Miller, B. A., Hristova, K., and Usherwood, P. N. (1993). Flexoelectric effects in model and native membranes containing ion channels. European biophysics journal: EBJ 22, 289-300.
Rinaldi, P. C., Jones, J. P., Reines, F., and Price, L. R. (1991). Modification by focused ultrasound pulses of electrically evoked responses from an in vitro hippocampal preparation. Brain Research 558, 36-42.
SHEALY, C. N., and HIENNEMAN, E. (1962). Reversible effects ofultrasound on spinal reflexes. Ar-chives of neurology 6, 374-386.
Sukharev, S., and Corey, D. P. (2004). Mechanosensitive channels: multiplicity of families and gating paradigms. Science&apos;s STKE: signal transduction knowledge environment 2004, re4.
ter Haar, G. (2007). Therapeutic applications of ultrasound. Progress in biophysics and molecular biology 93, 111-129.
Tsui, P.-H., Wang, S.-H., and Huang, C.-C. (2005). In vitro effects of ultrasound with different energies on the conduction properties of neural tissue. Ultrasonics 43, 560-565.
Tufail, Y., Matyushov, A., Baldwin, N., Tauchmann, M. L., Georges, J., Yoshihiro, A., Tillery, S. I. H., and Tyler, W. J. (2010). Transcranial pulsed ultrasound stimulates intact brain circuits. Neuron 66, 681-694.
Tufail, Y., Yoshihiro, A., Pati, S., Li, M. M., and Tyler, W. J. (2011). Ultrasonic neuromodulation by brain stimulation with transcranial ultrasound. Nature protocols 6, 1453-1470.
Tyler, W. J., Tufail, Y., Finsterwald, M., Tauchmann, M. L., Olson, E. J., and Majestic, C. (2008). Remote excitation of neuronal circuits using low-intensity, low-frequency ultrasound. PLoS ONE 3, e3511.
Veiling, V. A., and Shklyaruk, S. P. (1988). Modulation of the functional state of the brain with the aid of focused ultrasonic action. Neuroscience and behavioral physiology 18, 369-375.
Yang, T., Chen, 3., Yan, B., and Zhou, D. (2011). Transcranial ultrasound stimulation: a possible therapeutic approach to epilepsy. Medical Hypotheses 76, 381-383.
Yoo, S.-S., Kim, H., Min, B.-K., Franck, E., and Park, S. (2011). Transcranial focused ultrasound to the thalamus alters anesthesia time in rats. NeuroReport 22, 783-787.
Zaghi, S., Acar, M., Hultgren, B., Boggio, P. S., and Fregni, F. (2010). Noninvasive brain stimulation with low-intensity electrical currents: putative mechanisms of action for direct and alternating current stimulation. The Neuroscientist: a review journal bringing neurobiology, neurology and psychiatry 16, 285-307.

### BRIEF DESCRIPTION OF THE DRAWINGS

FIG. 1 shows ultrasonic-transducer targeting of the Orbito-Frontal Cortex (OFC), Anterior Cingulate Cortex (ACC), and Insula.

FIG. 2 shows a block diagram of the control circuit.

FIG. 3 shows a schematic diagram that defines terms related to a bioTU waveform with a pulsed ultrasound protocol.

FIG. 4 shows a schematic diagram that defines terms related to a bioTU waveform with a continuous ultrasound protocol.

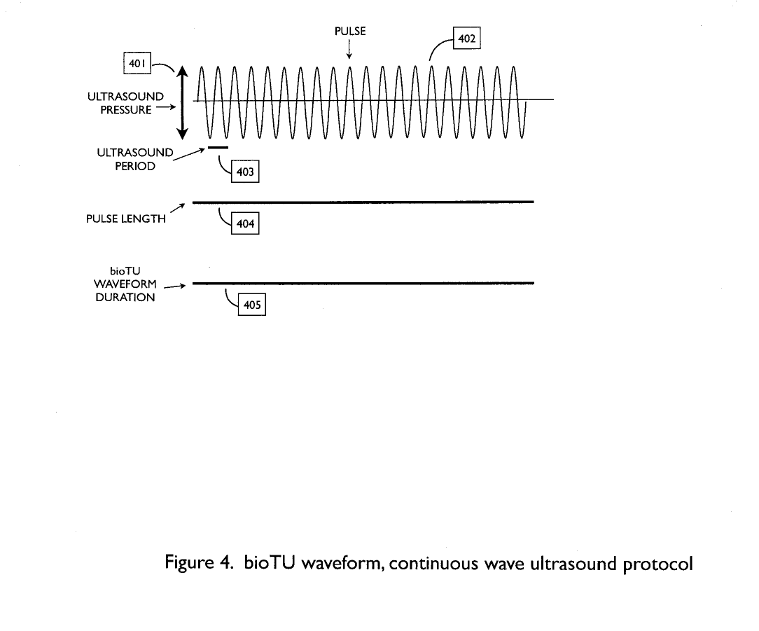

FIG. 5 shows a schematic diagram that defines terms related to bioTU waveform repetition.

### DETAILED DESCRIPTION OF THE INVENTION

Recent research and disclosures have described the use of transcranial ultrasound (bioTU) to activate, inhibit, or modulate neuronal activity (Bystritsky et al., 2011; Tufail et al., 2010; Tufail et al., 2011; Tyler et al., 2008; Yang et al., 2011; Yoo et al., 2011; Zaghi et al., 2010). bioTU protocols directed at the brain of a human or animal activate, inhibit, or modulate neuronal activity primarily through mechanical effects when delivered with the appropriate ultrasound waveform.

It is the purpose of this invention to provide methods and systems and methods for neuromodulation of deep-brain targets using ultrasound to treat depression, bipolar disorder, and other mood disorders. In some embodiments of the invention, methods and systems and methods for neuromodulation of deep-brain targets use ultrasound to affect the mood or emotional state of a subject or user. In various embodiments of the invention, modulation of one or a plurality of human emotions chosen from the following non-exhaustive list are chosen: Affection, Anger, Angst, Anguish, Annoyance, Apathy, Arousal, Awe, Boldness. Boredom, Contempt, Contentment, Curiosity, non-clinical Depression, Desire, Despair, Disappointment, Disgust, Dread, Ecstasy, Embarrassment, Envy, Euphoria, Excitement, Fear, Fearlessness, Frustration, Gratitude, Grief Guilt, Happiness, Hatred, Hope, Horror, Hostility, Hurt, Hysteria, Indifference, Interest, Jealousy, Joy, Loathing, Loneliness, Love, Lust, Misery, Passion, Pity, Pleasure, Pride, Rage, Regret, Remorse, Sadness, Satisfaction, Shame, Shock, Shyness, Sorrow, Suffering, Surprise, Terror, Wonder, Worry, Zeal, and Zest. Such neuromodulation systems can produce applicable acute or long-term effects. The latter occur through Long-Term Potentiation (LTP) and Long-Term Depression (LTD) via training. Included is control of direction of the energy emission, intensity, frequency, pulse duration, and phase/intensity relationships to targeting and accomplishing up-regulation and/or down-regulation.

bioTU is a beneficial new technique for modulating brain circuit activity via patterned, local vibration of brain tissue using US having an acoustic frequency greater than about 100 kHz and less than about 10 MHz. In common embodiments, ultrasound energy in a bioTU waveform is present at a range of acoustic frequencies in this range. bioTU transmits mechanical energy through the skull to its target in the brain without causing significant thermal or mechanical damage and induces neuromodulation. bioTU employs low intensity ultrasound such that the spatial-peak, temporal-average intensity (Iₛₚₜₐ) of the bioTU protocol is less than about 1 W/cm² in the targeted brain tissue. The acoustic intensity measure Iₛₚₜₐ is calculated according to established techniques well known to those skilled in the art that relate to the ultrasound acoustic pressure and other bioTU protocol characteristics such as the temporal average power during the bioTU waveform duration. US may be delivered as short-lived continuous waves less than about seconds, in a pulsed manner, or in the form of an ultrasound waveform of arbitrary complexity during bioTU protocols such that diverse patterns of neuromodulation can be delivered. For modulating the activity of brain circuits through localized tissue vibration, bioTU protocols may utilize US waveforms of any type known in the art. These include amplitude modulated waveforms, tone-bursts, pulsed waveforms, continuous waveforms, and other waveform patterns that will be described in detail below.

In a preferred embodiment of this invention, bioTU is used to induce neuromodulation in a subject whereby:
One or more ultrasound transducers are coupled to the head of an individual human or animal (the ‘subject’, ‘user’, or ‘recipient’);

1) Components of the bioTU device are near or wearably attached to the recipient in order to provide power and control the intensity, timing, targeting, and waveform characteristics of the transmitted acoustic waves;

2) a bioTU protocol is triggered that uses a waveform that:
a. has an acoustic frequency between about 100 kHz and about 10 MHz; and
b. has a spatial-peak, temporal-average intensity between about 0.0001 mW/cm² and about 1 W/cm²; and
c. does not induce heating of the brain due to bioTU that exceeds about 2 degrees Celsius for more than about 5 seconds.

3) the bioTU protocol induces an effect on neural circuits in one or more brain regions to treat depression, bipolar disorder, or another mood disorder; or to affect the mood or emotional state of a subject;

Ultrasound can be defined as low or high intensity (ter Haar, 2007). In contrast to bioTU, ultrasound imaging generally employs high frequency ultrasound (greater than about 1 MHz). In ultrasound, acoustic intensity is a measure of power per unit of cross sectional area (e.g. mW/cm²) and requires averaging across space and time. The intensity of the acoustic beam can be quantified by several metrics that differ in the method for spatial and temporal averaging. These metrics are defined according to technical standards established by the American Institute for Ultrasound in Medicine and National Electronics Manufacturers Administration (NEMA. Acoustic Output Measurement Standard For Diagnostic Ultrasound Equipment (National Electrical Manufacturers Association, 2004)). A commonly used intensity index is the ‘spatial-peak, temporal-average’ intensity (Iₛₚₜₐ). The intensities reported herein refer to Iₛₚₜₐ at the targeted brain region.

Acoustic frequencies greater than about 1 MHz used in ultrasound imaging and most previous ultrasound neuromodulation studies have disadvantages in regard to tissue heating and transmission of mechanical energy (Tsui et al., 2005). Damage due to ultrasound can occur due to thermal effects (heating) or mechanical effects (such as inertial cavitation—the creation of air bubbles that expand and contract with the time-varying pressure waves). High-intensity US can readily produce mechanical and/or thermal tissue damage (Dalecki, 2004; Hynynen and Clement, 2007; O'Brien, 2007; ter Haar, 2007), precluding it from use in non-invasive brain-circuit stimulation. High-intensity US (>1 W/cm²) influences neuronal excitability by producing thermal effects (Tsui et al., 2005). In addition to the initial studies cited above, high-intensity US has been reported to modulate neuronal activity in peripheral nerves (Mihran et al., 1990; Tsui et al., 2005), craniotomized cat and craniotomized rabbit cortex (Velling and Shklyaruk, 1988), peripheral somatosensory receptors in humans (Gavrilov et al., 1976), cat spinal cord (SHEALY and HENNEMAN, 1962), and rodent hippocampal slices (Bachtold et al., 1998; Rinaldi et al., 1991). While these prior studies support the general potential of US for neurostimulation, high-intensity US can readily produce mechanical and/or thermal tissue damage (Dalecki, 2004; Hynynen and Clement, 2007; O'Brien, 2007; ter Haar, 2007), precluding it from use in non-invasive brain-circuit stimulation. These studies delivered ultrasound directly to the brain or periphery. Transcranial delivery of ultrasound at these frequencies leads to tissue heating, particularly of bone in the skull.

Since low-frequency US can be reliably transmitted through skull bone (Hynynen and Clement, 2007; Hynynen et al., 2004) transcranial US is capable of safely and reliably stimulating in vivo brain circuits in humans and animals. Appropriate acoustic waveform protocols for neuromodulation without causing damage were recently discovered (Tufail et al., 2010; Tufail et al., 2011; Tyler et al., 2008). bioTU employs an ultrasound acoustic waveform that transmits mechanical energy through the skull to its target in the brain without causing damage. bioIT is an advantageous form of brain stimulation due to its non-invasiveness, safety, focusing characteristics, and the capacity to vary bioTU waveform protocols for specificity of neuromodulation.

US causes the local vibration of particles, leading to both mechanical and thermal effects. In some embodiments, bioTU brain stimulation protocols modulate neuronal activity primarily through mechanical means.

One important piece of evidence indicating that the mechanism of bioTU is primarily mechanical rather than thermal is that the timecourse of neuromodulation correlates more strongly with the timecourse of mechanical energy transmission than with the timecourse of thermal effects in the tissue. Tufail, Tyler, and colleagues showed that electrophysiological responses to bioTU in mice occur within tens to hundreds of milliseconds of the onset of the bioTU protocol. In contrast, tissue heating occurs on a timescale of 100s of milliseconds to seconds (Tufail et al., 2010). Moreover, effective bioTU brain stimulation occurred in these mice without tissue heating. In these studies, a 0.87 mm diameter thermocouple (TA-29, Warner Instruments, LLC, Hamden, Conn., USA) was inserted into motor cortex through a cranial window and no deviation in brain temperature greater than the noise level of these recordings (about 0.01 degrees Celsius) was observed (Tufail et al., 2010).

The mechanical effects of US induce neuromodulation before mechanical energy becomes absorbed to a degree such that sufficient tissue heating can occur to affect neural circuit function by thermal means. The acoustic pressure wave begins to affect the mechanosensitivity of lipid bilayers, protein channels, and neuronal membranes at the speed of sound in tissue (microseconds to tens of microseconds). The temporally lagging tissue heating incurred by US tends to be slower than the mechanical effects requiring tens of milliseconds or longer.

The thermal index (TI) of ultrasound is the ratio of power applied to that which would raise the temperature of tissue by I degree Celsius. The TI is an important parameter used to assess the heating of tissue due to absorption of energy from the acoustic waves. Bone absorbs ultrasound to a greater degree than other tissues, so TI values for bone are higher for a given ultrasound waveform relative to other tissues. The skull reflects, diffracts, and absorbs acoustic energy fields during transcranial US transmission. The acoustic impedance mismatches between the skin-skull and skull-brain interfaces present additional challenges ihr transmitting and focusing US through the skull into the intact brain. The absorption of ultrasound by bone is highly dependent on the acoustic frequency with more absorption at frequencies greater than about 1 MHz. Ultrasound below about 0.7 MHz is transmitted more effectively through bone and thus beneficial for bioTU due to reduced heating of the skull. A second reason that bioTU employs lower acoustic frequencies than used for imaging applications is that the mechanical index of ultrasound scales inversely with the square root of the acoustic frequency. Thus, reducing the acoustic frequency by half (e.g. from 1 M-Hz to 0.5 MHz) increases the mechanical power transmitted to the target tissue by about 1.4 (the square root of 2).

The parameters of bioTU are critical for ensuring that neuromodulation occurs without damage. bioTU parameters, described in more detail below, include the use of low intensity (less than about 1 W/cm² at the target tissue), low acoustic frequency (between about 100 kHz and about 10 MHz), and an appropriate pulse repetition frequency, pulse length, waveform duration, and other waveform parameters such that the temperature of the target brain region does not rise by more than about 2 degrees Celsius for a period longer than about 5 seconds. In some specific embodiments, a single pulse is delivered that may be referred to as a continuous wave (CW) pulse by one skilled in the art and extends in time for about longer than 10 ms, about longer than 100 ms, about longer than 1 second, or any length of time up to and including 5 seconds. Complex bioTU waveforms, including bioTU waveforms generated by hybridization, convolution, addition, subtraction, phase shifting, concatenation, and joining with an overlap for a portion of each of the waveforms for two or more bioTU waveforms or bioTU waveform components, as well as modulation or ramping of the intensity of all or a portion of the waveform, or modulation or ramping of any other parameter used to define an ultrasound waveform, would be advantageous for bioTU.

Appropriate bioTU protocols are advantageous for mitigating or eliminating tissue damage while simultaneously modulating neuronal activity primarily through mechanical means. For example, low temporal average intensity can be achieved by reducing the acoustic power of the ultrasound waves or by varying one or more bioTU parameters to decrease the effective duty cycle—the proportion of time during a bioTU waveform that ultrasound is delivered. Reduced duty cycles can be achieved by decreasing one or more bioTU parameters chosen from pulse length, cycles per pulse, pulse repetition frequency, or other waveform parameters. Low temporal average intensity can be achieved by varying one or more ultrasound parameters during a bioTU protocol. For instance, the acoustic power may be decreased during a portion of a bioTU protocol. Alternatively, the pulse repetition frequency can be increased during a bioTU protocol. In other embodiments, complex ultrasound waveforms can be generated that are effective for inducing neuromodulation and maintain an appropriately low temporal average intensity.

Depending on the bioTU protocol, activation or inhibition of brain activity can be achieved (Yoo et al., 2011). Although not intending to be restricted to any one theory for the activation of voltage-gated channels by bioTU, one hypothesis for opening of these channels is by mechanical stretching of the receptors to an open configuration. In alternative embodiments, alternate bioTU stimulation protocols can be chosen in order to specifically activate one or more types of membrane bound, cytoskeletal, or cytoplasmic proteins including ion channels, ion pumps, or secondary messenger receptors. In this embodiment, it would be possible to selectively activate or inhibit specific cell types based on their expression of the targeted protein.

A bioTU protocol delivers ultrasound to one or more brain regions and induces neuromodulation that correlates more strongly in time with the timecourse of mechanical effects on tissue than thermal effects. The dominant acoustic frequency for bioTU is generally greater than about 100 kHz and less than about 10 MHz. In common embodiments of bioTU, a mix of acoustic frequencies are transmitted. Particularly advantageous acoustic frequencies are between about 0.3 MHz and 0.7 MHz. The spatial-peak temporal-average (Iₛₚₜₐ) intensity of the ultrasound wave in brain tissue is greater than about 0.0001 mW/cm² and less than about 1 W/cm². Particularly advantageous Iₛₚₜₐ values are between about 100 mW/cm² and about 700 mW/cm². The Iₛₚₜₐ value for any particular bioTU protocol is calculated according to methods well known in the art that relate to the ultrasound pressure and temporal average of the bioTU waveform over its duration. Effective ultrasound intensities for activating neurons or neuronal circuits do not cause tissue heating greater than about 2 degrees Celsius for a period longer than about 5 seconds.

Significant attenuation of ultrasound intensity occurs at the boundaries between skin, skull, dura, and brain due to impedance mismatches, absorption, and reflection so the required ultrasound intensity delivered to the skin or skull may exceed the intensity at the targeted brain region by up to 10-fold or more depending on skull thickness and other tissue and anatomical properties.

Providing a mixture of ultrasound frequencies is useful for efficient brain stimulation. Various strategies for achieving a mixture of ultrasound frequencies to the brain of the user are known. Driving an ultrasound transducer at a frequency other than the resonant frequency of the transducer is one way to create ultrasound waves that contain power in a range of frequencies. For instance, an ultrasound transducer with a center frequency of 0.5 MHz can be driven with a sine wave at 0.35 MHz. A second strategy for producing ultrasound waves that contain power in a range of frequencies is to use square waves to drive the transducer. A third strategy for generating a mixture of ultrasound frequencies is to choose transducers that have different center frequencies and drive each at their resonant frequency. A fourth strategy for generating a mixture of ultrasound frequencies is to drive an ultrasound transducer with a waveform that itself contains multiple frequency components. One or more of the above strategies or alternative strategies known to those skilled in the art for generating US waves with a mixture of frequencies would also be beneficial.

Mixing, amplitude modulation, or other strategies for generating more complex bioTU waveforms can be beneficial for driving distinct brain wave activity patterns or to bias the power, phase, or spatial extent of brain oscillations such as slow-wave, delta, beta, theta, gamma, or alpha rhythms.

The effect of bioTU on brain activity may be increased or decreased by the action of at least one of the ultrasound waves, which may include increasing or decreasing neuron firing, receptivity, release or uptake of neurohormones, neurotransmitters or neuromodulators, increase or decrease of gene transcription, protein translation or protein phosphorylation or cell trafficking of proteins or mRNA, or affect the activity of other brain cell or brain structure activity.

The major advantages of bioTU for brain stimulation are that it offers a mesoscopic spatial resolution of a few millimeters and the ability to penetrate beyond the brain surface while remaining completely non-invasive. bioTU has beneficial advantages over other forms of non-invasive neuromodulation that include focusing, targeting tissues at depth, and painless stimulation procedures. Ultrasound also offers a rich degree of flexibility for modifying the stimulation protocol. One potentially advantageous aspect of the large parameter space available for bioTU is the possibility of improving the specificity of the induced neuromodulation effect with regard to cell type, sub-cellular compartment, receptor type, or brain structure by varying bioTU parameters. In contrast, other non-invasive forms of brain stimulation are more limited in the extent to which stimulation parameters can be varied. For instance, the spatial extent of TMS is fixed for a given electromagnet. For tDCS, only the location and type of electrodes, current amplitude, and stimulus duration can be varied. Due to its rich parameter space for being able to generate a wide variety of distinct stimulus wavefbrms yielding different effects on neural activity patterns (Tufail et al., 2011), bioTU is well-suited for non-invasive brain stimulation.

In some embodiments, bioTU can be delivered from a phased array of transducers for improved targeting of one or more brain regions. Constructive and destructive interference of acoustic waves transmitted by multiple transducers can be used to deliver complex spatiotemporal patterns of acoustic waves. Moreover, the spectral density of acoustic pressure profiles delivered to a targeted brain region can be varied to produce differential effects on neuronal activity. These properties of bioTU offer the possibility of activating widely distributed brain networks. In certain embodiments, the capacity to target distributed brain regions concurrently or with a specific order further extends the possibilities for modulating brain activity. In an alternative embodiment, a plurality of ultrasound transducers are employed for delivering bioTU to a subject and the bioTU waveform delivered from some or all ultrasound transducers differs in one or a plurality of parameters that may include intensity, acoustic frequency, pulse duration, pulse repetition frequency, or another parameter that defines the bioTU waveform.

The dominant acoustic frequency used for bioTU is one parameter that determines the induced neuromodulatory effect. In advantageous embodiments of the invention, the dominant acoustic frequency is generally greater than about 100 kHz and less than about 10 MHz. Particularly advantageous acoustic frequencies are between about 0.3 MHz and 0.7 MHz. The spatial-peak temporal-average (Iₛₚₜₐ) intensity of the ultrasound waveform at the site of cells to be modulated is less than about 1 WX/cm². Particularly advantageous Iₛₚₜₐ values are between about 100 mW/cm² and about 700 mW/cm² at the site of the cells to be modulated. In some embodiments of the invention, the pulse repetition frequency for inhibition is 300 Hz or lower (depending on condition and patient). In some embodiments of the invention, the pulse repetition frequency for excitation is in the range of 500 Hz to 5 MHz. In some embodiments of the invention, the ultrasound acoustic frequency is in range of 0.3 MHz to 0.8 MHz with power generally applied less than 60 mW/cm² but also at higher target- or patient-specific levels at which no tissue damage is caused. In some embodiments of the invention, the acoustic frequency is gated at the lower rate to impact the neuronal structures as desired (e.g., say 300 Hz for inhibition (down-regulation) or 1 kHz for excitation (up-regulation). Ultrasound therapy can be combined with therapy using other devices (e.g., Transcranial Magnetic Stimulation (TMS), deep-brain stimulation (DBS), application of optogenetics, radiosurgery, Radio-Frequency (RF) therapy, transcranial direct current stimulation (tDCS), or other brain stimulation technologies) and/or medications.

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. Ultrasound conduction medium will be required to fill the space.

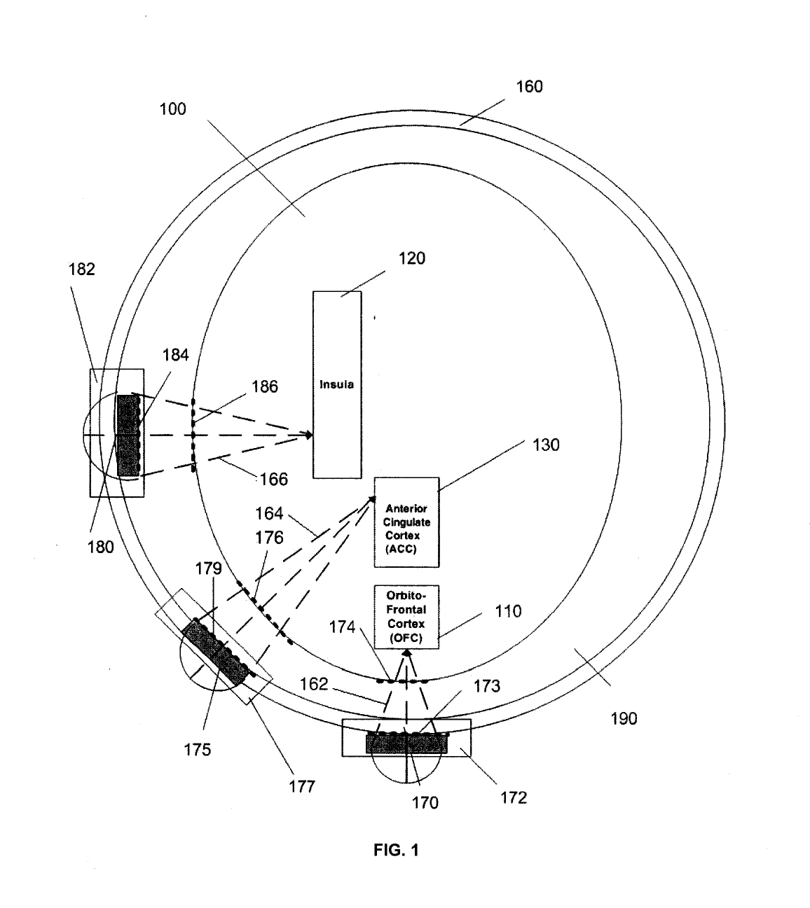

FIG. 1 shows a set of ultrasound transducers targeting to treat depression and bipolar disorder. The head 100 contains the three targets, Orbito-Frontal Cortex (OFC) 110, Insula 120, and Anterior Cingulate Cortex (ACC) 130. These targets are hit by ultrasound from transducers 170 with ultrasound beam 162, 175 with ultrasound beam 164, and 180 with ultrasound beam 166, with their respective holders 172, 177, and 182 fixed to track 160. Ultrasound transducer 170 is shown targeting the OFC 110, transducer 175 is shown targeting the ACC 130, and transducer 180 is shown targeting the Insula 120. Transducer 170 is moved radially in or out of holder 172 and fixed into position. In like manner, transducer 175 is moved radially in or out of holder 177 and fixed into position and transducer 180 is moved radially in or out of holder 182 and fixed into position. In other embodiments, transducers 170, 175, and 180 are directly fixed on track 160. For ultrasound to be effectively transmitted to and through the skull and to brain targets, coupling must be put into place. Ultrasound transmission (for example Dermasol from California Medical Innovations) medium 190 is interposed with one mechanical interface to the ultrasound transducers 170, 175, 180 (completed by a layers of ultrasound transmission gel 173, 179, 184) and the other mechanical interface to the head 100 (completed by a layers of ultrasound transmission gel 174, 176, 186). This figure shows a fixed configuration where the appropriate radial (in-out) positions have determined through patient-specific imaging (e.g., PET or fMRI) and the holders positioning the ultrasound transducers are fixed in the determined positions. To support this embodiment, treatment-planning software is used taking the image-determined target positions and output instructions for manual or computer-aided manufacture of the holders. Alternatively positioning instructions can be output for the operator to position the blocks holding the transducers to be correctly placed relative to the support track. In one embodiment, the transducers positioned using this methodology can be aimed up or down and/or left or right for correct flexible targeting.

Transducer array assemblies of this type may be supplied to custom specifications by Imasonic in France (e.g., large 2D High Intensity Focused Ultrasound (HIFU) hemispheric array transducer) (Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Keramos-Etalon in the U.S. is another custom-transducer supplier. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations.

Several strategies are known for targeting bioTU to a specific brain region. When using water-matched transducers, the transmission of US from the transducer into the brain only occurs at points at which acoustic gel (or other coupling fluid) physically couples the transducer to the head. On the basis of this acoustic transmission property, coupling the transducer to the head through small gel contact points represents one physical method for transmitting US into restricted brain regions (Tufail et al., 2010). In this embodiment, the entire face of the transducer should always be covered with acoustic gel to prevent heating and damage of the transducer lace. The area of gel coupling the transducer to the head, however, can be sculpted to restrict the lateral extent through which US is transmitted into the brain. Although this method does provide an effective approach for stimulating coarsely targeted brain regions, calculating acoustic intensities transmitted into the brain with this method can be difficult because of nonlinear variations in the acoustic pressure fields generated.

Alternatively, the lateral extent of the spatial envelope of US transmitted into the brain can be restricted by using acoustic collimators. Single-element transducers having concave focusing lenses or transducers shaped to deliver a targeted acoustic wave can also be used for delivering focused acoustic pressure fields to brains. Such single-element focused transducers can be manufactured having various focal lengths depending on the lens curvature, as well as the physical size and center frequency of the transducer. The most accurate yet complicated US focusing method involves the use of multiple transducers operating in a phased array.

An appropriate ultrasound stimulation protocol must be delivered in order to induce changes in the brain via bioTU. The temporal pattern of ultrasound vibration delivered to the brain affects the induced neuromodulation. The temporal pattern of ultrasound waveforms may also affect the nature of the induced neuromodulatory effect such as neuromodulation (which may be mediated by a change in the excitability of neuronal circuits), stimulation of neuronal activity, inhibition of neuronal activity, or modulation of one or a plurality of the following biophysical or biochemical processes: (i) ion channel activity, (ii) ion transporter activity, (iii) secretion of signaling molecules, (iv) proliferation of the cells, (v) differentiation of the cells, (vi) protein transcription of cells, (vii) protein translation of cells, (viii) protein phosphorylation of the cells, or (ix) protein structures in the cells. In some embodiments, bioTU may induce different effects concurrently in different brain regions. In some embodiments, bioTU may induce effects in non-targeted brain regions.

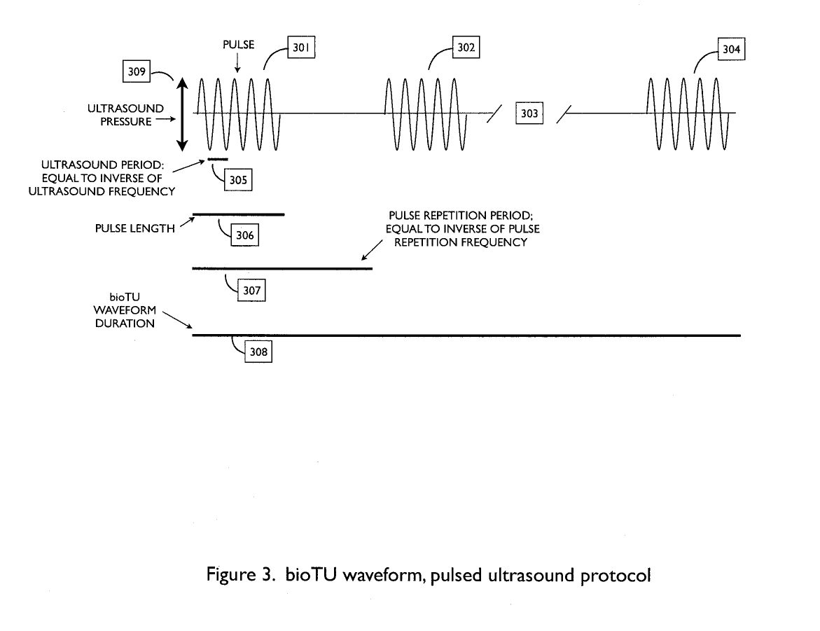

Pulsing of ultrasound is an effective strategy for activating neurons that reduces the temporal average intensity while also achieving desired brain stimulation or neuromodulation effects. In addition to acoustic frequency (305) and transducer variables, several waveform characteristics such as cycles per pulse, pulse repetition frequency, number of pulses, and pulse length affect the intensity characteristics and outcome of any particular bioTU stimulus on brain activity. A pulsed bioTU protocol generally uses pulse lengths (306) between about 0.5 microseconds and about 1 second. A bioTU protocol may use pulse repetition frequencies (PRFs) between about 50 Hz and about 25 kHz (307). Particularly advantageous PRFs are generally between about 1 kHz and about 3 kHz. For pulsed bioTU waveforms, the number of cycles per pulse (cpp) is between about 5 and about 10,000,000. Particularly advantageous cpp values vary depending on the choice of other bioTU parameters and are generally between about and about 250. The number of pulses for pulsed bioTU waveforms is between about 1 pulse and about 125,000 pulses. In FIG. 3, the 1st (301), 2nd (302), and nth (304) pulses are shown, with the gap in the horizontal line (303) indicating additional pulses that may number between about 1 and about 125,000 pulses. In this embodiment, the number of pulses defines the bioTU waveform duration (308). In some embodiments, particularly advantageous pulse numbers for pulsed bioTU waveforms are between about 100 pulses and about 250 pulses.

Tone bursts that extend for about 1 second or longer—though, strictly speaking, also pulses—are often referred to as continuous wave (CW). In alternative embodiments, one or more continuous wave (CW) ultrasound waveforms less than about five seconds in duration (401, 402, 403, 404, 405) is directed to the brain to induce neuromodulation. US protocols that include such CW waveforms offer advantages for neuromodulation due to their capacity to drive activity robustly. However, one disadvantage of bioTU protocols with CW pulses is that the temporal average intensity is significantly higher which may cause painful thermal stimuli on the scalp or skull and may also induce heating and thus damage in brain tissue. Thus, advantageous embodiments using CW pulses may employ a lower acoustic intensity and/or a slow pulse repetition frequency of less than about 1 Hz. For instance, a CW US stimulus waveform with 1 second pulse lengths repeated at 0.5 Hz would deliver US every other second. Alternative pulsing protocols including those with slower pulse repetition frequencies of less than about 0.5 Hz or less than about 0.1 Hz or less than about 0.01 Hz or less than about 0.001 Hz are also beneficial. In some useful embodiments, the interval between pulses or pulse length may be varied during a bioTU protocol that includes CW pulses.

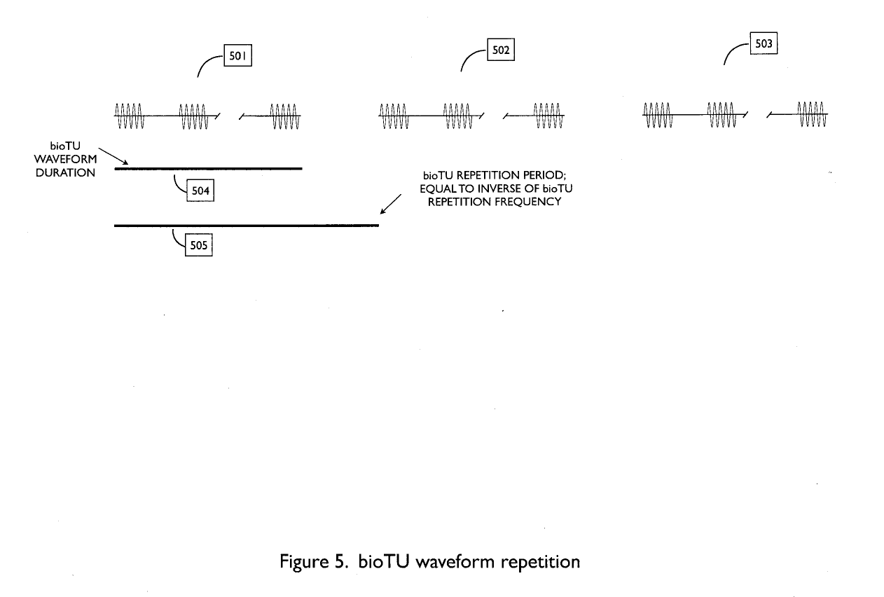

In some embodiments, repeating the bioTU protocol is advantageous for achieving particular forms of neuromodulation. In some embodiments, the number of times a bioTU protocol of appropriate duration (504) is repeated is chosen to be in the range between 2 times and 100,000 times. FIG. 5 (501, 502, 503) presents a schematic of three repeated bioTU protocols. Particularly advantageous numbers of bio TU protocol repeats are between 2 and 1,000 repeats. The bioTU repetition frequency (505) of a bioTU protocol may be less than about 10 Hz, less than about 1 Hz, less than about 0.1 Hz, or lower. The bioTU repetition frequency may be fixed or variable. Variable bioTU repetition frequency values may be random, pseudo-random, ramped, or otherwise modulated. The bioTU repetition period is defined as the inverse of the bioTU repetition frequency.

Effective and ineffective parameters for ultrasound neuromodulation have been described previously (e.g. (Tufail et al., 2010; Tyler et al., 2008), patent application Ser. Nos. 13/003,853 (Publication number: US 2011/0178441 A1) titled “Methods and devices for modulating cellular activity using ultrasound” and PCT/US20110/055527 (Publication number: WO/2011/057,028) titled “Devices and methods for modulating brain activity” by inventor Tyler).

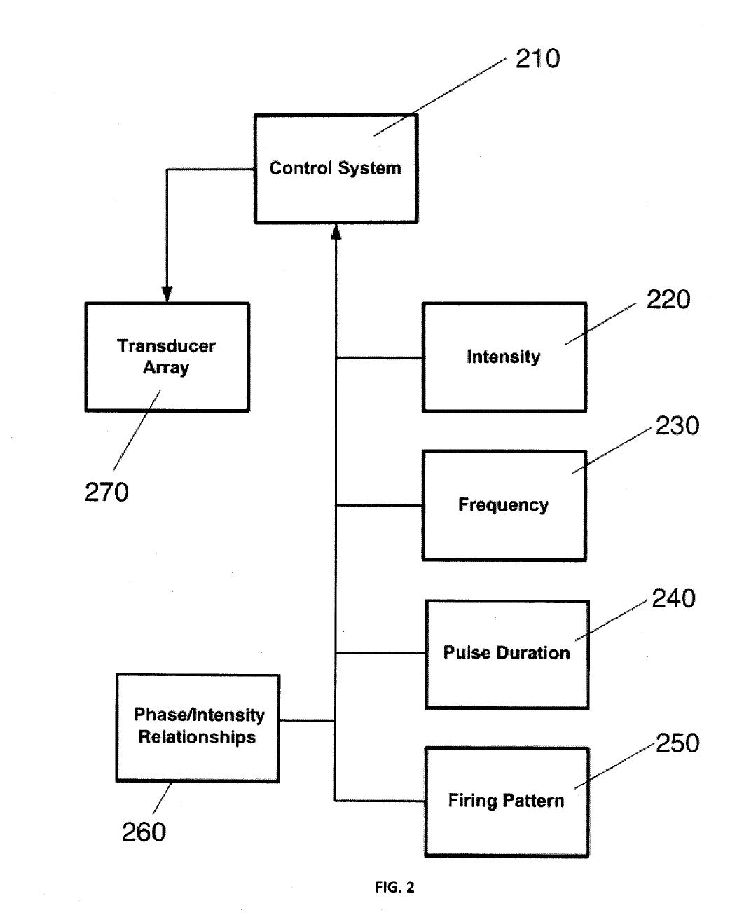

FIG. 2 shows an embodiment of a control circuit. The positioning and emission characteristics of transducer array 270 are controlled by control system 210 with control input with neuromodulation characteristics determined by settings of intensity 220, frequency 230, pulse duration 240, firing pattern 250, and phase/intensity relationships 260 for beam steering and focusing on neural targets.

In another embodiment, a feedback mechanism is applied such as functional Magnetic Resonance Imaging (fMRI), Positive Emission Tomography (PET) imaging, video-electroencephalogram (V-EEG), acoustic monitoring, thermal monitoring, other form of physiological monitoring, and/or feedback from the patient or user.

In still other embodiments, other energy sources are used in combination with or substituted for ultrasound transducers that are selected from the group consisting of Transcranial Magnetic Stimulation (TMS), deep-brain stimulation (DBS), optogenetics application, radiosurgery, Radio-Frequency (RF) therapy, and medications.

The invention allows stimulation adjustments in variables such as, but not limited to, intensity, firing pattern, frequency, pulse duration, phase/intensity relationships, dynamic sweeps, and position.

The invention incorporates hardware and software components for generating ultrasound protocols of arbitrary complexity. Complex wavefbrms can be generated by any technique known in the art for generating control signals for driving one or a plurality of ultrasound transducers and related components. In most embodiments, voltage-varying waveforms will be generated by dedicated software and/or hardware.

In some embodiments of the invention, ultrasound waveforms are generated algorithmically using one or a plurality of mathematical equations. In some embodiments, combinatorial techniques are used to generate bioTU waveforms. In alternative embodiments, bioTU waveforms are generated by adding, subtracting, hybridizing, concatenating, convolving, or otherwise combining two or more bioTU waveforms or bioTU waveform components. In common embodiments, bioTU waveforms may take the form of pulse trains of ultrasound. According to these various embodiments, pulse trains may similarly be generated by adding, subtracting, hybridizing, concatenating, convolving, or otherwise combining two or more bioTU pulse trains. Triggering is an effective and simple strategy for generating a variety of bioTU waveforms. In some embodiments, multiplying and dividing bioTU waveforms or bioTU waveform components is used to generate complex bioTU waveforms. In alternative embodiments of the invention, multiple bioTU waveforms or bioTU waveform components are combined with temporal offsets and/or voltage offsets. In yet other embodiments, a combination of more than one method for generating bioTU waveforms is used, such as a combination of triggering and adding, subtracting, hybridizing, concatenating, convolving, or otherwise combining two or more bioTU waveforms. For instance, a bioTU waveform can be generated by triggering a particular bioTU waveform or bioTU waveform component upon the occurrence of a threshold crossing event of another slower sinusoidal waveform.

Previous disclosures concerning ultrasound neuromodulation have described continuous and pulsed waveforms. As disclosed in patent application Ser. No. 13/003,853 (Publication number: US 2011/0178441 A1) by inventor Tyler titled “Methods and devices for modulating cellular activity using ultrasound”, an ultrasound pulse may be generated by brief bursts of square waves, sine waves, saw-tooth waveforms, sweeping waveforms, or arbitrary waveforms, or combinations of one or more waveforms. The waveforms may be focused or not focused. The method may be repeated. The components for generating ultrasound, such as ultrasound transducer or its elements, are driven using analog or digitized waveforms. Ultrasound transducer elements may be driven using individual waveforms or a combination of square, sine, saw-tooth, or arbitrary waveforms. As further disclosed in patent application PCT/US2010/055527 (Puiblication number: WO/2011/057028) by inventor Tyler titled “Devices and methods for modulating brain activity”, ultrasound pulses for bioTU may be sine waves having a single ultrasound frequency, other oscillating shapes may be used, such as square waves, or spikes, or ramps, or a pulse includes multiple ultrasound frequencies composed of beat frequencies, harmronics, or a combination of frequencies generated by constructive or deconstructive interference techniques, or some or all of the aforementioned. As disclosed in patent application Ser. No. 13/098,473 (Publication number: US 2011/0270138) titled “Ultrasound Macro Pulse And Micro Pulse Shapes For Neuromodulation”, individual pulses can be shaped by superimposing pulse trains on the base ultrasound carrier and heterogeneous patterns of pulse shaping with sine waves, square waves, triangular waves, or arbitrarily shaped waves.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

### DEFINITIONS

In this application, we use the terms ‘brain stimulation’, ‘neuromodulation’, and ‘neuronal activation’ interchangeably to refer to invasive or non-invasive techniques to alter the excitability, action potential rate, vesicular release rate, or other biochemical pathway in neurons or other cell types in the brain.

In this application we use the terms “bioTU”, “bioTU protocol”, ‘bioTU stimulation protocol’, ‘bioTU stimulation waveform’, ‘ultrasound stimulation protocol’, ‘ultrasound stimulation waveform,” and “bioTU stimulation” interchangeably to refer a modulation of brain circuit activity induced by patterned, local vibration of brain tissue using US whereby:

Ultrasound is transmitted into the brain;

A dominant acoustic frequency is generally greater than about 100 kHz and less than about 10 MHz. Particularly advantageous acoustic frequencies are between about 0.3 MHz and 0.7 MHz;

The spatial-peak temporal-average (Iₛₚₜₐ) intensity of the ultrasound waveform at the brain tissue is less than about 1 W/cm². Particularly advantageous Iₛₚₜₐ values are between about 100 mW/cm² and about 700 mW/cm².

The ultrasound pulse length is less than about 5 seconds; and

The protocol induces an effect in one or more brain regions such as neuromodulation, brain activation, neuronal activation, neuronal inhibition, or a change in blood flow whereby heating of brain tissue does not exceed approximately 2 degrees Celsius for a period greater than about 5 seconds.

In this application, we define mechanical effects of ultrasound waves in the brain as effects caused by the local vibration of brain tissue. We define thermal effects of ultrasound waves in the brain as effects caused by the heating of brain tissue.

In this application, we define the term “pulse length” as the amount of time of a non-interrupted tone burst of one or more ultrasound acoustic wave frequency components.

In this application, we define the term “pulse repetition period” to be the amount of time between the onset of consecutive ultrasound pulses. The “pulse repetition frequency” is equivalent to the inverse of the “pulse repetition period”.

In this application, we define the term “bioTU waveform” to be a period of ultrasound delivered with a pulsed or continuous wave construction or more complex waveform. bioTU waveforms may be that includes a specified number of pulses that may be repeated at the pulse repetition frequency. In some cases, a bioTU waveform is composed of a single continuous wave tone burst of greater than about one second that is not repeated. In such cases, the “pulse length” and “bioTU waveform duration” may be about equal.

In this application, we define the term “bioTU waveform component” to be a feature of a bioTU waveform that, in isolation, is insufficient to fully define a bioTU waveform.

In this application, we define the term “bioTU repetition period” to be the amount of time of between the onset of consecutive bioTU waveforms. The “bioTU repetition frequency” is equivalent to the inverse of the “bioTU repetition period”.

As used in the specification and the appended claims, the singular forms “a,” “an,” and “the” include plural referents unless the context clearly dictates otherwise. Thus, for example, reference to “an ultrasound waveform” includes mixtures of two or more ultrasound waveforms, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occurs and that the description includes instances where said event or circumstance occurs and instances where it does not. The term “treating” refers to inhibiting, preventing, curing, reversing, attenuating, alleviating, minimizing, suppressing or halting the deleterious effects of a disease and/or causing the reduction, remission, or regression of a disease. Those of skill in the art will understand that various methodologies and assays can be used to assess the development of a disease, and similarly, various methodologies and assays may be used to assess the reduction, remission or regression of the disease.

“Increase” is defined throughout as less than a doubling such as an increase of 5%, 10%, or 50% or as an increase of 1, 2, 3, 4, 5, 6, 7, 8, 9, 10, 11, 12, 13, 14, 15, 16, 17, 18, 19, 20 21, 23, 24, 25, 26, 27, 28, 29, 30, 31, 32, 33, 34, 35, 36, 37, 38, 39, 40, 41, 42, 43, 44, 45, 46, 47, 48, 49, 50 51, 52, 53, 54, 55, 56, 57, 58, 59, 60, 61, 62, 63, 64 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 400, or 500 times increase as compared with basal levels or a control.

What is claimed is:

1. A method for stimulating deep-brain tissue to cause neuromodulation, the method comprising:
focusing a plurality of ultrasound transducer at one or a plurality of neural targets which affect target regions of the brain, and
applying pulsed power to the ultrasound transducer(s); and
controlling the pulsed power to affect a mood disorder of the patient, whereby the mood disorder is alleviated.

2. The method of claim 1, wherein:
a. ultrasound is transmitted into the brain at a plurality of ultrasound transducers targets one or a plurality of brain regions related to an emotional state or mood disorder;
b. the dominant acoustic frequency is greater than about 100 kHz and less than about 10 MHz;
c. the spatial-peak temporal-average (Iₛₚₜₐ) intensity of the ultrasound waveform at the site of cells to be modulated is less than about 1 W/cm²;
d. the ultrasound pulse length is less than about 5 seconds; and
e. the transmitted ultrasound induces an effect in one or more brain regions such as neuromodulation, brain activation, neuronal activation, neuronal inhibition, or a change in blood flow whereby heating of brain tissue does not exceed approximately 2 degrees Celsius for a period greater than about 5 seconds.

3. The method whereby the mood disorder treated is depression.

4. The methods of claim 1, whereby the mood disorder treated is bipolar disorder.

5. The methods of claim 1, whereby the mood disorder treated is a mood disorder other than depression or bipolar disorder.

6. The methods of claim 1 whereby the affected emotional state is one or a plurality of Affection, Anger, Angst, Anguish, Annoyance, Apathy, Arousal, Awe, Boldness, Boredom, Contempt, Contentment, Curiosity, Non-Clinical Depression, Desire, Despair, Disappointment, Disgust, Dread, Ecstasy, Embarrassment, Envy, Euphoria, Excitement, Fear, Fearlessness, Frustration, Gratitude, Grief, Guilt, Happiness, Hatred, Hope, Horror, Hostility, Hurt, Hysteria, Indifference, Interest, Jealousy, Joy, Loathing, Loneliness, Love, Lust, Misery, Passion, Pity, Pleasure, Pride, Rage, Regret, Remorse, Sadness, Satisfaction, Shame, Shock, Shyness, Sorrow, Suffering, Surprise, Terror, Wonder, Worry, Zeal, or Zest.

7. The method of claim 1, further comprising aiming an ultrasound transducer neuromodulating neural targets related to the group consisting of depression and bipolar disorder in a manner selected from the group of up-regulation, down-regulation.

8. The method of claim 1, wherein the effect is chosen from the group consisting of acute and long term, including Long-Term Potentiation and Long-Term Depression.

9. The methods of claim 1, wherein one or a plurality of targets are selected from the group consisting of Pre-Frontal, Orbito-Frontal Cortex, Anterior Cingulate Cortex, Subgenu Cingulate, Insula, Nucleus Accumbens, Caudate Nucleus, Amygdala, and Hippocampus.

10. The methods of claim 1, wherein the acoustic ultrasound frequency is in the range of 0.3 MHz to 0.8 MHz.

11. The method of claim 1, wherein a stimulation frequency for of approximately 500 Hz or lower is applied for inhibition of neural activity.

12. The method of claim 11 wherein modulation frequency of lower than approximately 500 Hz is divided into pulses 0.1 to 20 msec. repeated at frequencies of 2 Hz or lower for down regulation.

13. The method of claim 1, wherein the stimulation frequency for excitation is in the range of 500 Hz to 5 MHz.

14. The method of claim 13 wherein modulation frequency of approximately 500 Hz or higher is divided into pulses 0.1 to 20 msec. repeated at frequencies higher than 2 Hz for up regulation.

15. The method of claim 1, where in the power applied is less than 60 mW/cm².

16. The method of claim 1, wherein the power applied is greater than 60 mW/cm² but less than that causing tissue damage.

17. The method of claim 1, wherein the focus area of the pulsed ultrasound is 0.5 to 50 mm in diameter.

18. The method of claim 1, wherein the focus area of the pulsed ultrasound is 0.5 to 50 mm in diameter.

19. The method of claim 1, wherein the focus area of the pulsed ultrasound is 50 to 150 mm in diameter.

20. The method of claim 1, wherein the number of ultrasound transducers is between 1 and 10.

21. The method of claim 1, wherein mechanical perturbations are applied radially or axially to move the ultrasound transducers.

22. The method of claim 1, wherein a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, thermal r mornitoring, and patient feedback.

23. The method of claim 1, wherein ultrasound therapy is combined with or replaced by one or more therapies selected from the group consisting of Transcranial Magnetic Stimulation (TMS), deep-brain stimulation (DBS), transcranial Direct Current Stimulation (tDCS), application of optogenetics, radiosurgery, Radio-Frequency (RF) therapy, and medications.
